(19) United States
(12) Patent Application Publication
(10) Pub. No.: US 2025/0183440 A1
(43) Pub. Date: Jun. 5, 2025

(54) APPARATUS HAVING BATTERY ACCOMMODATION UNIT

(71) Applicants: RAYENCE Co., Ltd., Gyeonggi-do (KR); VATECH EWOO Holdings Co., Ltd., Gyeonggi-do (KR)

(72) Inventors: Jung Do KIM, Gyeonggi-do (KR); Yong Wook PARK, Gyeonggi-do (KR)

(73) Assignees: RAYENCE Co., Ltd., Gyeonggi-do (KR); VATECH EWOO Holdings Co., Ltd., Gyeonggi-do (KR)

(21) Appl. No.: 18/844,276

(22) PCT Filed: Feb. 1, 2023
(86) PCT No.: PCT/KR2023/001453

§ 371 Date: Sep. 5, 2024

(30) Foreign Application Priority Data
Mar. 7, 2022 (KR) 10-2022-0028684

(51) Int. Cl. H01M 50/24; G01T 1/175; H01M 50/247; H01M 50/262; H01M 50/271

(52) U.S. Cl. CPC H01M 50/24; G01T 1/175; H01M 50/247; H01M 50/262; H01M 50/271; H01M 2220/30

(57) ABSTRACT

The present invention relates to an apparatus having a battery accommodation unit capable of coupling/releasing a battery cover in a one-touch manner, the battery cover covering a battery accommodation groove and pressing a packing. Also, according to the present invention, a hook is arranged on an outskirt region of the packing surrounding the edge of the battery accommodation groove, So that sealing of the battery accommodation groove, as well as the coupling/releasing of the battery cover, can be simultaneously implemented.

### TECHNICAL FIELD

The present disclosure relates to an apparatus having a battery accommodation unit capable of coupling/releasing a battery cover, which covers a battery accommodation groove and presses a seal, in a one-touch manner.

### BACKGROUND ART

Many products used in our daily lives come in the form of a battery storage device where a battery is attached to and detached from a main body of the battery storage device. As an example of such a battery storage device, mobile X-ray detectors that can be freely moved are increasingly being utilized in medical or industrial settings.

A battery weighing hundreds of grams is attached to and detached from a mobile X-ray detector, and the battery is generally attached to and detached from the main body using a cover with a lock pin structure.

For this purpose, a space for installing a battery is provided on the back surface of the main body, and when the battery is installed in the battery installation space, the battery installation space is covered with the separate cover and the cover and the main body are connected with lock pins.

When using a lock pin like this, a user needs to directly rotate the lock pins 90 degrees using a fastening means (a driver, a wrench, etc.).

Additionally, it takes time to attach and detach the battery because multiple lock pins must be rotated.

In addition, because rotating multiple lock pins equally is difficult, the binding force may be different, and thus there is a risk that the lock pins may be released arbitrarily.

Moreover, as multiple lock pins are disposed inside the main seal of the battery, additional seals for the lock pins are also required.

### DOCUMENT OF RELATED ART

### Patent Document

(Patent Document 1) Korean Patent Application Publication No. 10-2021-0122985

### DISCLOSURE

### Technical Problem

The present disclosure is intended to solve the above problems occurring in the related art. An objective of the present disclosure is to provide an apparatus having a battery accommodation unit capable of easily coupling/releasing a battery cover in a one-touch manner and sealing (waterproofing and dustproofing) with only a seal that surrounds a battery.

### Technical Solution

In order to achieve the above-mentioned objectives, there is provided an apparatus having a battery accommodation unit, the apparatus including: a main body in which a battery accommodation groove is formed; a battery cover configured to cover the battery accommodation groove; a seal configured to surround an edge of the battery accommodation groove or an edge of the battery cover; and a hook frame provided in the main body, disposed outside at least one edge of the battery accommodation groove and movable by manipulation by a user, and equipped with one of a plurality of hooks or hook grooves, wherein the battery cover may cover the battery accommodation groove and press the seal, and may be provided with remaining one of the plurality of hooks or hook grooves that is selectively coupled to/released from the one of the plurality of hooks or hook grooves by moving the hook frame.

### Advantageous Effects

According to an apparatus having a battery accommodation unit of the present disclosure, the following effects are achieved.

Since a hook is placed on the outskirt of a seal surrounding the edge of a battery accommodation groove, not only can a battery cover be coupled/released, but the battery accommodation groove can be sealed at the same time.

Furthermore, it is very easy to assemble as the coupling is done by simply pressing the battery cover in a one-touch manner.

Furthermore, since the battery cover can be released with one touch due to a return elastic body, it is very easy to remove the battery cover.

### DESCRIPTION OF DRAWINGS

FIGS. 1 and 2 are separated and combined perspective views showing an apparatus having a battery accommodation unit according to an embodiment of the present disclosure.

FIGS. 3 to 6 are perspective views showing the assembly sequence of FIG. 2.

FIG. 7 is an enlarged perspective view of a hook frame of FIG. 1.

FIGS. 8 and 9 are front and rear perspective views showing a battery cover of FIG. 1.

### BEST MODE

Embodiments disclosed in the present specification will be described in detail with reference to the attached drawings, but identical or similar components will be assigned the same reference numerals regardless of the numbers used in the drawings to identify the components, and redundant descriptions thereof will be omitted. The terms “module” and “part” for components used in the following description are given or used interchangeably only for the ease of preparing the specification, and do not have distinct meanings or roles in themselves. In addition, in describing the embodiments disclosed in this specification, if it is determined that detailed descriptions of related known technologies may obscure the gist of the embodiments disclosed in this specification, the detailed description will be omitted. In addition, the attached drawings are only for easy understanding of the embodiments disclosed in this specification, and the technical idea disclosed in this specification is not limited by the attached drawings. The present disclosure is not limited to the embodiments, but should be understood to include all changes, equivalents, and substitutes included in the spirit and technical scope of the present disclosure.

Terms containing ordinal numbers, such as first, second, etc., may be used to describe various components, but the components are not limited by the terms. The above terms are used solely for the purpose of distinguishing one component from another.

When a component is said to be “connected” or “coupled” to another component, it should be understood that the component may be directly connected to or coupled to the other component, but that other components may exist in between. On the other hand, when a component is said to be “directly connected” or “directly coupled” to another component, it should be understood that there are no other components in between.

Singular expressions include plural expressions unless the context clearly dictates otherwise.

It should be further understood that the terms “comprise”, “include”, “have”, etc., when used in this specification, specify the presence of stated features, integers, steps, operations, elements, components, and/or combinations thereof but do not preclude the presence or addition of one or more other features, integers, steps, operations, elements, components, and/or combinations thereof.

In the drawings, the sizes of components may be exaggerated or reduced for convenience of explanation. For example, the size and thickness of each component shown in the drawings are arbitrarily shown for convenience of explanation, and thus the present disclosure is not necessarily limited to what is shown.

In cases where an embodiment may be implemented differently, a specific process sequence may be performed differently from the described sequence. For example, the two processes described in succession may be performed substantially at the same time, or may be performed in the reverse order from the order in which they are described.

Meanwhile, since the present disclosure may be divided into several embodiments depending on the specific configuration and operation, each will be examined separately below. For convenience of explanation, common content for each embodiment is sufficiently explained through a first embodiment, and other embodiments are explained mainly on differences from the first embodiment.

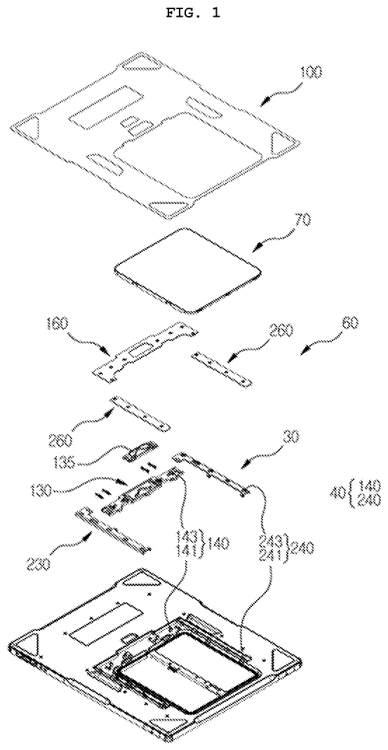

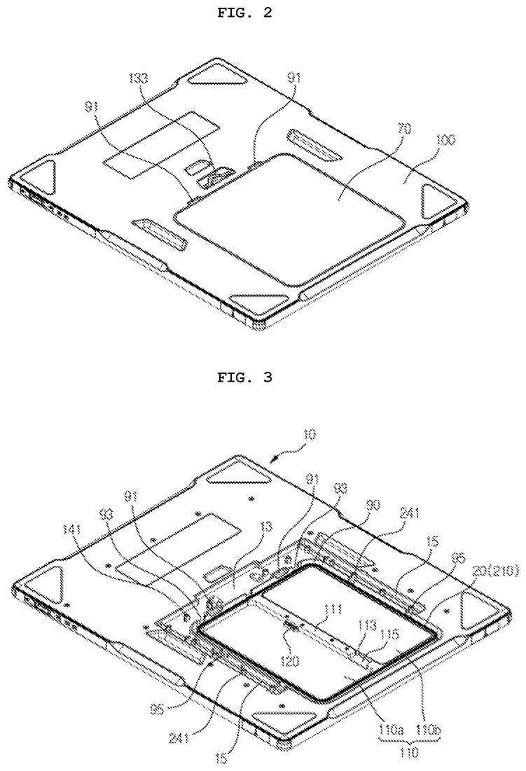

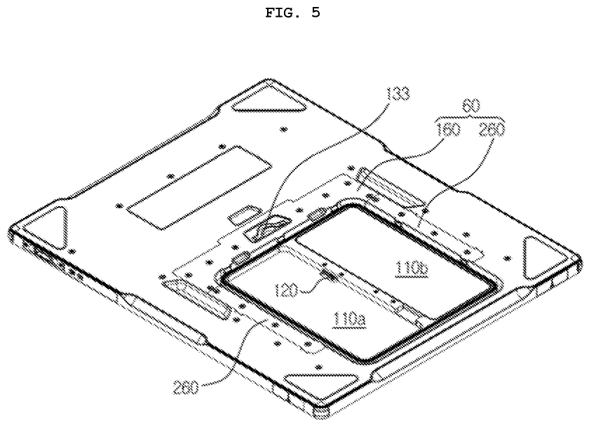

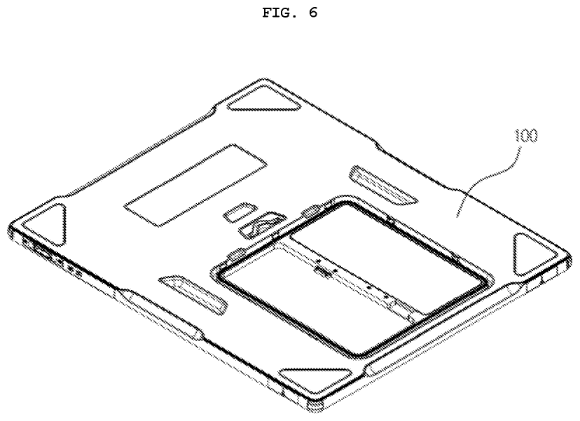

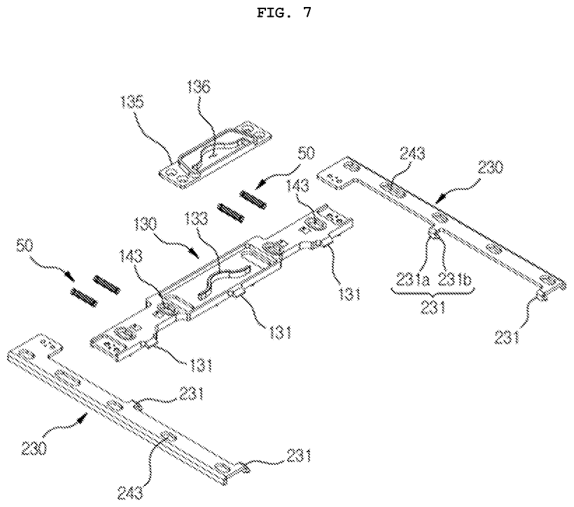

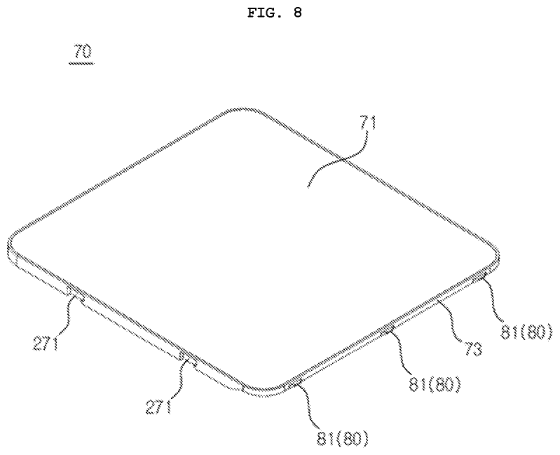

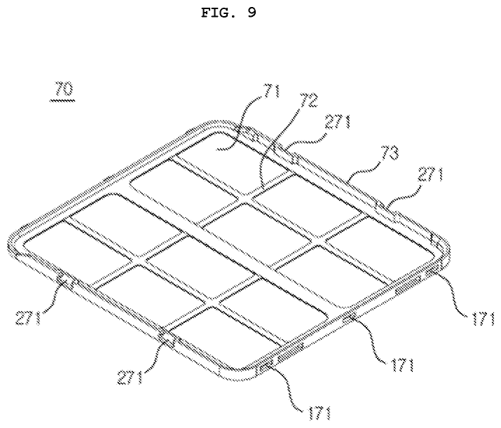

FIGS. 1 and 2 are separated and combined perspective views showing an apparatus having a battery accommodation unit according to an embodiment of the present disclosure, FIGS. 3 to 6 are perspective views showing the assembly sequence of FIG. 2, FIG. 7 is an enlarged perspective view of a hook frame of FIG. 1, and FIGS. 8 and 9 are front and rear perspective views showing a battery cover of FIG. 1.

Referring to FIGS. 1 to 6, an apparatus 1 having a battery accommodation unit according to an embodiment of the present disclosure may include a main body 10. The main body 10 may have a rectangular parallelepiped shape with a thin thickness.

Referring to FIG. 3, the main body 10 may include a battery accommodation groove 110. The battery accommodation groove 110 may be formed on the back surface of the main body 10. The battery accommodation groove 110 may have a square shape. The battery accommodation groove 110 may be divided into two left and right halves. The battery accommodation groove 110 may include first and second battery accommodation grooves 110a and 110b. The first and second battery accommodation grooves 110a and 110b may be partitioned by a partition 111. A communication groove 113 may be formed in the partition 111. The communication groove 113 may be a part of the partition 111 cut out. The communication groove 113 may communicate with the first and second battery accommodation grooves 110a and 110b. An undercut groove 115 may be formed below the communication groove 113. The bottom of the undercut groove 115 may be in a deeper position than the bottom of the first battery accommodation groove 110a or the bottom of the second battery accommodation groove 110b. Thus, when removing a battery from the first and second battery accommodation grooves 110a and 110b, a user inserts his or her fingers into the communication groove 113 and the undercut groove 115 and lifts one side of the battery. The battery may be mounted horizontally in the battery accommodation groove 110.

The main body 10 may include a connector 120. The connector 120 may be placed in the battery accommodation groove 110. When the battery is mounted in the battery accommodation groove 110, the connector 120 may receive power from the battery. For example, when the main body 10 is a mobile X-ray detector, a sensor panel (not shown), a panel drive board (not shown), a main board (not shown), etc. may be mounted inside the main body 10. The connector 120 may be electrically connected to the main board (not shown), and the main board (not shown) may be electrically connected to the panel drive board (not shown).

The apparatus 1 having a battery accommodation unit according to an embodiment of the present disclosure may include a seal 20. The seal 20 may be mounted in a seal mounting groove 210 formed in the main body 10. The seal mounting groove 210 may be shaped to surround the outer edge of the battery accommodation groove 110. Thus, when the seal 20 is mounted on the seal mounting groove 210, the seal 20 may have a closed-loop sealed shape surrounding the outer edge of the battery accommodation groove 110. When a battery cover 70 is closed, the seal 20 may be elastically deformed in all directions, that is, forward, backward, left and right. The seal 20 may be installed by implementing a seal mounting groove on the back surface of the battery cover 70, which will be described later, instead of the battery accommodation groove 110.

FIG. 7 is an enlarged perspective view of a hook frame of FIG. 1.

Referring to FIG. 7, the apparatus 1 having a battery accommodation unit according to an embodiment of the present disclosure may include a hook frame 30. The hook frame 30 may be installed in the main body 10.

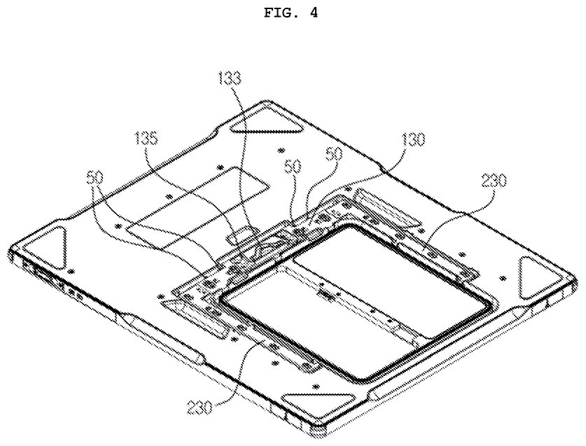

The hook frame 30 may include a first hook frame 130, which is the front hook frame. The first hook frame 130 may have a long strip shape left and right. The first hook frame 130 may be disposed on the outer edge of the front edge of the battery accommodation groove 110. The first hook frame 130 may be movably mounted on a first sliding groove 13. The first sliding groove 13 may be formed on the outer edge of the front edge, which is one side of the battery accommodation groove 110 of the main body 10. Referring to FIG. 7, the first hook frame 130 may include a first hook 131. The first hook 131 may be formed to protrude from the rear end of the first hook frame 130. The first hook 131 may have a cross-section perpendicular to the longitudinal direction to be configured as a right triangle. That is, the upper surface of the first hook 131 may be formed as the hypotenuse. The hypotenuse may facilitate forward sliding of the first hook frame 130 when the battery cover 70 is closed. The first hook frame 130 may include a user operation button 133. The user operation button 133 may be shaped like a strip protruding upward from one side of the first hook frame 130. The user operation button 133 may be easily pushed and pulled by the user with a finger. Referring to FIG. 4, the first hook frame 130 may include a user operation button cover 135. The user operation button cover 135 is a cover body having an exposure hole 136 through which the user operation button 133 is exposed. The user operation button cover 135 may be fixed to the main body 10. Thus, the exposure hole 136 may guide the forward and backward movement of the user operation button 133.

The hook frame 30 may include a second hook frame 230, which is left and right hook frames, respectively connected to the left and right sides of the first hook frame 130. Each second hook frame 230 may be L-shaped. The second hook frame 230 may be disposed on the outside of the left and right edges corresponding to the left and right sides of the front edge, which is one side of the battery accommodation groove 110. The second hook frames 230 may be movably mounted on second sliding grooves 15, respectively. The second sliding groove 15 may be formed on the left and right outer edges of the battery accommodation groove 110 of the main body 10. The upper sides of the second hook frames 230 are respectively fixed to the left and right sides of the first hook frame 130, and may move together. Referring to FIG. 7, the second hook frame 230 may include a second hook 231. The second hook 231 may be formed to protrude from the inside of the second hook frame 230. The second hook 231 may include a 2ahook 231a protruding from the inside of the second hook frame 230 and a 2b hook 231b protruding from the rear of the 2a hook 231a. The 2b hook 231b may first act as a pusher when the battery cover 70 is closed. The upper surfaces of the 2a hook 231a and the 2b hook 231b may be formed as the hypotenuse. The hypotenuse may facilitate forward sliding of the second hook frame 230.

The apparatus 1 having a battery accommodation unit according to an embodiment of the present disclosure may include a guide part 40. The guide part 40 may guide the forward and backward movement of the hook frame 30.

The guide part 40 may include a first guide part 140. The first guide part 140 may guide the forward and backward movement of the first hook frame 130.

The first guide part 140 may include a first guide protrusion 141. The first guide protrusion 141 may be formed in the first sliding groove 13.

The first guide part 140 may include a first guide elongated hole 143. The first guide elongated hole 143 may be formed back and forth on the first hook frame 130. The first guide elongated hole 143 may be slidably fitted into the first guide protrusion 141.

The guide part 40 may include a second guide part 240. The second guide part 240 may guide the forward and backward movement of the second hook frame 230.

The second guide part 240 may include a second guide protrusion 241. The second guide protrusion 241 may be formed in the second sliding groove 15.

The second guide part 240 may include a second guide elongated hole 243. The second guide elongated hole 243 may be formed back and forth on the second hook frame 230. The second guide elongated hole 243 may be slidably fitted into the second guide protrusion 241.

The apparatus 1 having a battery accommodation unit according to an embodiment of the present disclosure may include a return elastic body 50. The return elastic body 50 may be implemented, for example, as a compression coil spring. The return elastic body 50 may return the movement of the hook frame 30 with respect to the main body 10. That is, when the user pulls the hook frame 30 forward, the battery cover 70 is unlocked, and when the user releases the hook frame 30, the hook frame 30 may be returned to the original position thereof.

The apparatus 1 having a battery accommodation unit according to an embodiment of the present disclosure may include a hook frame cover 60. The hook frame cover 60 may cover the first and second sliding grooves 13 and 15 to cover the hook frame 30 so that the hook frame 30 may be moved within the grooves 13 and 15. The hook frame cover 60 may include a first hook frame cover 160 covering the first sliding groove 13 and a second hook frame cover 260 covering the second sliding groove 15. The first hook frame cover 160 and the second hook frame cover 260 may be fastened to the first guide protrusion 141 and the second guide protrusion 241.

FIGS. 8 and 9 are front and rear perspective views showing a battery cover of FIG. 1.

Referring to FIGS. 8 and 9, the apparatus 1 having a battery accommodation unit according to an embodiment of the present disclosure may include the battery cover 70. The battery cover 70 may cover the battery accommodation groove 110 and pressurize the seal 20. That is, the battery cover 70 and the seal 20 may perform a waterproof function to seal the battery accommodation groove 110 and protect the battery accommodation groove 110 from external moisture. The battery cover 70 may be composed of a rectangular plate.

The battery cover 70 may include a top plate 71, and a side plate 73 extending downward along the edge of the top plate 71. A grid-shaped rib 72 may be formed on the bottom surface of the top plate 71. The rib 72 may serve to press the seal 20 and the battery. The rib 72 may be pressed into linear contact with the battery to reduce the contact area with the battery cover 70. The reduction in contact area may cause the battery cover 70 to easily separate from the battery when released. First hook grooves 171 may be formed on the front surface of the side plate 73. The first hook grooves 171 may be selectively coupled to/disengaged from the first hook 131. Second hook grooves 271 may be formed on the left and right sides of the side plate 73. The second hook grooves 271 may be selectively coupled to/disengaged from the second hook 231.

The apparatus 1 having a battery accommodation unit according to an embodiment of the present disclosure may include a battery cover pivoting part 80. The battery cover pivoting part 80 may rotate the battery cover 70 with respect to one side of the main body 10 to conveniently close the battery cover 70.

The battery cover pivoting part 80 may include a pivoting protrusion 81 formed on the rear surface of the side plate 73 and a pivoting groove (not shown) formed on the main body 10. After the pivoting protrusion 81 is inserted into the pivoting groove (not shown), the battery cover 70 may be engaged in a one-touch manner by closing the battery cover 70. Thus, the pivoting groove (not shown) and the pivoting protrusion 81 not only support the pivoting of the battery cover 70 but also form one axis of engagement/disengagement.

Disengagement of the battery cover 70 may be done when the user operation button 133 is pulled forward (up), the hook frame 30 moves upward, which causes the first and second hooks 131 and 231 to fall out of the first and second hook grooves 171 and 271.

Referring to FIG. 3, the outer surface of the side plate 73 of the battery cover 70 is surrounded by a fence 90 of the main body 10. The fence 90 may be placed on the outside of the seal mounting groove 210. The side plate 73 may be inserted between the fence 90 and the seal mounting groove 210. At this time, a concave groove 91 may be formed in front of the fence 90. The concave groove 91 may be a space for lifting the battery cover 70 with a finger. Additionally, a first communication groove 93 may be formed in front of the fence 90. The first communication groove 93 may be U-shaped. The first communication groove 93 may be arranged so that the first hook 131 can slide. In addition, a second communication groove 95 may be formed on each of the left and right sides of the fence 90. The second communication groove 95 may also be U-shaped. The second communication groove 95 may be arranged so that the second hook 231 can slide.

The apparatus 1 having a battery accommodation unit according to an embodiment of the present disclosure may include a finishing plate 100. The finishing plate 100 may finish parts excluding the battery cover 70.

In the above description, it was explained that the hook is provided on the hook frame and the hook groove is provided on the battery cover, but the opposite may be true. That is, even if the hook frame is provided with one of the hook or the hook groove, and the battery cover is provided with the other of the hook or the hook groove, the same role can be expected. In addition, it was explained that the pivoting protrusion is provided on the battery cover and the pivoting groove is provided on the main body, but the opposite may be true. That is, even if the battery cover is provided with one of the pivoting protrusion or the pivoting groove, and the main body is provided with the other of the pivoting protrusion or the pivoting groove, the same role can be expected.

Any or other embodiments of the present disclosure described above are not exclusive or distinct from each other. In certain embodiments or other embodiments of the present disclosure described above, each configuration or function may be used in combination or combined.

It is obvious to those skilled in the art that the present disclosure may be embodied in other specific forms without departing from the spirit and essential features of the present disclosure. The above detailed description should not be construed as limiting in any respect and should be considered illustrative. The scope of the present disclosure should be determined by reasonable interpretation of the appended claims, and all changes within the equivalent scope of the present disclosure are included in the scope of the present disclosure.

### Description of Reference Numerals

1: apparatus having battery accommodation unit 10: main body
110: battery accommodation groove 120: connector
20: seal 210: seal mounting groove
30: hook frame 40: guide part
50: return elastic body 60: hook frame cover
70: battery cover 80: battery cover pivoting part
90: fence 100: finishing plate

1. An apparatus having a battery accommodation unit, the apparatus comprising:
a main body in which a battery accommodation groove is formed:
a battery cover configured to cover the battery accommodation groove:
a seal configured to surround an edge of the battery accommodation groove or an edge of the battery cover; and
a hook frame provided in the main body, disposed outside at least one edge of the battery accommodation groove and movable by manipulation by a user, and equipped with one of a plurality of hooks or hook grooves,
wherein the battery cover covers the battery accommodation groove and presses the seal, and is provided with remaining one of the plurality of hooks or hook grooves that is selectively coupled to/released from the one of the plurality of hooks or hook grooves by moving the hook frame.

2. The apparatus of claim 1, wherein the plurality of hook grooves is coupled to/released from the plurality of hooks in a one-touch manner.

3. The apparatus of claim 2, further comprising:
a return elastic body configured to return movement of the hook frame relative to the main body.

4. The apparatus of claim 3, further comprising:
a user operation button provided on the hook frame and operated by the user.

5. The apparatus of claim 4, wherein the hook frame comprises:
a front hook frame provided with the user operation button; and
left and right hook frames configured to move together with the front hook frame, respectively connected to left and right sides of the front hook frame, and disposed on left and right outer edges of at least one edge of the battery accommodation groove.

6. The apparatus of claim 1, further comprising:
one of a pivoting protrusion or a pivoting groove provided on a second edge of the main body facing at least one edge of the battery accommodation groove of the main body, and remaining one of the pivoting protrusion or the pivoting groove provided on a side of the battery cover corresponding to the second edge of the main body and coupled to the one of the pivoting protrusion or the pivoting groove, so that the battery cover rotates based on the second edge of the main body to cover the battery accommodation groove and the seal.

7. The apparatus of claim 6, which is an X-ray detector mounted on the main body and further comprising a sensor panel for detecting X-rays.
